(19) United States
(12) Patent Application Publication
(10) Pub. No.: US 2013/0041454 A1
(43) Pub. Date: Feb. 14, 2013

(54) Sensor Actuated Stent

(71) Applicants: Melissa K. Dobson, Owego, NY (US); Nche A. Zama, Stroudsburg, PA (US)

(72) Inventors: Melissa K. Dobson, Owego, NY (US); Nche A. Zama, Stroudsburg, PA (US)

(73) Assignee: BUSINESS EXPECTATIONS LLC, Owego, NY (US)

(21) Appl. No.: 13/370,303

(22) Filed: Feb. 9, 2012

Related U.S. Application Data
(60) Provisional application No. 61/440,936, filed on Feb. 9, 2011.

(51) Int. Cl. A61F 2/82

(52) U.S. Cl. 623/1.15

(57) ABSTRACT

The invention is an implantable medical device to expand a vascular lumen into various positions, internally or from a remote location. The device is designed as a stent having a framework for radial or longitudinal expansion, including one or more integrated shape memory materials. The shape memory materials, or halos, radially expand to fix the framework into a first radial position. Sensors integrated with the framework mechanically or electro-mechanically monitor and control the positioning of the framework to a fixed second position, or gradually expand the framework to various radial positions. Visualization and communications devices assist in the monitoring and controlling mechanisms to position the implantable device during surgery or catherization, post-surgery, or at follow-up. The device is biocompatible, alleviating complications. The device can be utilized as a substitute, or in combination with another stent. Devices may be utilized in cardiovascular, neurovascular surgery or other intervention.

### RELATED APPLICATIONS

This Nonprovisional application claims benefit under 35 U.S.C. §119 to U.S. Provisional Patent Application Ser. No. 61/440,936 filed on Feb. 9, 2011 and titled Sensor Actuated Stent, which is incorporated herein by reference in its entirety.

### FIELD OF THE INVENTION

The present invention relates generally to implantable devices for interventional therapeutic treatment or vascular surgery and more particularly concerns expansion adjustments for a remote controlled stent.

### BACKGROUND OF THE INVENTION

The art and science of interventional therapy and surgery has continually progressed towards treatment of internal defects and diseases by use of ever smaller incisions to reduce the trauma to tissue surrounding the treatment site. One important aspect of such treatments involves the use of catheters to place therapeutic devices at a treatment site by access through the vasculature. Examples of such procedures include transluminal angioplasty, placement of stents to reinforce the walls of a blood vessel or the like and the use of vasocclusive devices to treat defects in the vasculature. A constant drive by those practicing in the art is to develop new and more capable systems for such applications. When coupled with developments in biological treatment capabilities, there is an expanding need for technologies that enhance the performance of interventional therapeutic devices and systems.

Coronary artery disease (CAD) is the leading cause of death among men and women in the United States, accounting for approximately one of every five deaths. Approximately 2.4 million adults have a history of myocardial infarction (MI), angina, or both, and it is estimated that millions of others have clinically silent coronary artery disease.

The traditional view has been that myocardial ischemia in CAD results from atherosclerotic plaques that narrow the vessel's lumen and decrease myocardial blood supply. Research has shown, however, that the reduction of blood flow results from the combination of fixed vessel narrowing and abnormal vascular tone, contributed to by atherosclerosis induced endothelial cell dysfunction.

Atherosclerotic coronary arteries can be treated by several methods. The treatment methods are divided into two major categories: non-invasive and invasive. Non-invasive treatments of coronary artery disease involve both medication and reduction of controllable risk factors of atherosclerosis. Non-invasive treatments cannot improve the coronary circulation when symptoms associated with coronary heart disease are severe. In such cases, invasive treatments are required to improve blood flow to the heart muscle. The most common invasive treatments for coronary heart disease include coronary artery bypass surgery (CABG), Percutaneous Transluminal Coronary Angioplasty (PTCA) and the use of percutaneously introduced prosthetic devices, namely, coronary stents. In CABG, a saphenous vein is removed from the patient's leg and sewn into the blood vessels that supply oxygenated blood to the heart. This transplanted vein, known as a graft, carries blood to the heart muscle, bypassing atherosclerotic areas in the coronary arteries. Alternatively, an internal mammary artery can be directly anastomosed distal to the stenotic site. CABG has become the most common major operation in the United States. It is usually a safe procedure with a ten-year patency rate of approximately 80-90%. Coronary bypass surgery effectively relieves chest pain (angina pectoris), increases exercise capacity, improves heart function in some patients, and prolongs life in certain patients. With current techniques, CABG may require one-week hospitalization.

In PTCA, the lumen of an atherosclerotic coronary artery is increased by the inflation of an intravascular balloon catheter. Today, balloon angioplasty is used successfully to treat atherosclerosis in both systemic (peripheral, renal, cerebral) and coronary arteries in selected patients. The success rate of PTCA by an experienced staff ranges from 80-95%, and the average hospital stay after PTCA is roughly two days. Advantages of PTCA include decreased hospital stay, lower cost, decreased recuperation time, and no requirement of general anesthesia or chest incision. Although these advantages have contributed to PTCA becoming a widely used alternative to CABG, the underlying mechanisms of balloon angioplasty continue to be elucidated.

PTCA was originally thought to increase the lumen by compacting the artheromatous material within a relatively unyielding artery. Histological studies indicate, however, that the mechanisms of balloon angioplasty are much more complex; two primary mechanism of the procedure are: (a) “overstretching” of the vascular wall, which causes some mural injury, and (b) plaque fracture with partial separation from the vascular wall. This localized damage of the arterial wall is essential for a successful dilation via balloon angioplasty. Nevertheless, it is assumed that excessive damage likely contributes to complications such as restenosis, dissection, vasospasm, and rupture. Although this vascular wall damage results from mechanical forces, it is not known at which level of mechanical stress or strain it occurs.

Specifically, one limitation associated with PTCA is the closure of the vessel, which may occur immediately after the procedure or during restenosis, gradual re-narrowing of the artery following the procedure. Additionally, restenosis is a chronic problem in patients who have undergone saphenous vein bypass grafting. The mechanism of acute occlusion appears to involve several factors and may result from vascular recoil with resultant closure of the artery and/or deposition of blood platelets and fibrin along the damaged length of the newly opened blood vessel.

Restenosis after PTCA is a more gradual process initiated by vascular injury. Multiple processes, including thrombosis, inflammation, growth factor and cytokine release, cell proliferation, cell migration and extracellular matrix synthesis each contribute to the restenotic process. Although the exact mechanism of restenosis is not completely understood, the general aspects of the process have been identified. In the normal arterial wall, smooth muscles cells proliferate at a low rate, approximately less than 0.1 percent per day. Smooth muscle cells in the vessel walls exist in a contractile phenotype characterized by eighty to ninety percent of the cell cytoplasmic volume occupied with the contractile apparatus. Endoplasmic reticulum, Golgi, and free ribosomes are few and are located in the perinuclear region. Extracellular matrix surrounds the smooth muscle cells and is rich in heparin-like glycosylaminoglycans, which are believed to be responsible for maintaining smooth muscle cells in the contractile phenotypic state (U.S. Pat. No. 7,806,924B2). Upon pressure expansion of an intracoronary balloon catheter during angioplasty, smooth muscle cells and endothelial cells within the vessel wall become injured, initiating a thrombotic and inflammatory response. Cell derived growth factors such as platelet derived growth factor, basic fibroblast growth factor, epidermal growth factor, thrombin, etc. released from platelets, invading macrophages and/or leukocytes, or directly from the smooth muscle cells, provoke a proliferative and migratory response in medial smooth muscle cells. These cells undergo a change from the contractile phenotype to a synthetic phenotype characterized by only a few contractile filament bundles, extensive rough endoplasmic reticulum, Golgi, and free ribosomes. Proliferation, migration, and extracellular matrix synthesis continue until the damaged endothelial layer is repaired at which time proliferation slows within the intima, usually within seven to fourteen days post-injury. The newly formed tissue is called neointima. The further vascular narrowing that occurs over the next three to six months is due primarily to negative or constrictive remodeling.

Simultaneous with local proliferation and migration, inflammatory cells adhere to the site of vascular injury. Within three to seven days post-injury, inflammatory cells have migrated to the deeper layers of the vessel wall.

Other risks associated with PTCA include blood clot formation within the stents even weeks or months after the angioplasty, leading to heart attack (Mayo Clinic). Bleeding at the sites of intervention (e.g. site of catheter insertion) may also lead to surgical intervention.

The limitations of PTCA cited above have resulted in the development of new technologies including atherectomy, laser angioplasty and coronary stenting.

The most significant change in interventional cardiology has been the growth of coronary stents, endovascular scaffolding devices that maintain the luminal integrity of diseased blood vessels. A typical stent implantation involves pre-dilatation of the target lesion via a PTCA procedure followed by implantation of the coronary stent in the same area of the coronary artery.

Unlike systemic pharmacologic therapy, stents have proven useful in reducing restenosis. Typically, stents are balloon-expandable slotted metal tubes (usually, but not limited to, stainless steel). The stents are implanted by mounting the stent on a balloon portion of a balloon catheter, positioning the stent within the lumen of a vessel [in a contracted state] and expanding the stent by inflating the balloon in order to maintain patency of the vessel. The balloon is then deflated and removed, leaving the stent in place. The expanded stent within the lumen of the angioplastied coronary artery provides structural support through rigid scaffolding to the arterial wall. This support is helpful in maintaining patency of the vessel lumen.

Modifications of the stents have been made to alleviate problems indicated above. Heparin coating of stents appears to have the added benefit of producing a reduction in sub-acute thrombosis after stent implantation. Thus, sustained mechanical expansion of a stenosed coronary artery with a stent has been shown to provide some measure of restenosis prevention, along with the clinical feasibility of delivering drugs locally to the site of injured tissue.

The placement including inflation and deflation of the balloon catheter is a complicate procedure that involves additional risks beyond the implantation of the stent, such that it would be desirable to provide a device that can be more simply placed in the site to be treated and less invasive to the lumen of the vessel. It is desirable to have an implantable device that adjusts and expands according to the patient's physiological needs.

A number of stents formed from polymeric memory materials are known to transform from a compressed configuration to an expanded configuration. One such conventional stent is known, for example, that provides a casing formed from a memory elastomer such as polyurethane, and a support structure that can be manufactured by braiding individual threads formed of temperature-sensitive polyurethane that is hard below 25° C. and that softens about 35° C. Thus, at a temperature slightly below body temperature, the stent changes from a pressed configuration to an expanded configuration.

The problem, however, is that stents formed of shape memory polymeric materials typically do not provide adequate structural and mechanical radial strength requirements for a stent. Stents are therefore commonly provided with a metallic structure to provide the strength required to function as a stent. It would therefore be desirable to provide a shape memory polymer or alloy having a configuration that would provide adequate structural and mechanical radial strength for a stent, and that can be situated without requiring inflation and deflation of a balloon catheter, and without injuring the inner vessel wall during expansion of the stent. An adjustable metallic structure in combination with a shape memory polymer would be desirable to remotely operate a stent to control its gradual expansion within a body lumen, both during and post-implantation, including a minimally invasive procedure (e.g. office visit). Sensor controls in combination with the stent would allow for monitoring of fluid flow through a luminary space, as well as visualization of the vessel, and automatic control and monitoring mechanisms to alert a health provider as to the status of the patient's implant. The invention will desirably be motorized on a micro-scale (e.g. by Micro-Electro-Mechanical Systems, “MEMS”), or nano-scale, for gradual expansion of a vessel. The present invention meets these and other needs. The new implantable systems, devices, and procedures may avoid many of the problems currently associated with stents.

### SUMMARY OF THE INVENTION

The following invention is an implantable medical device to expand a vascular lumen. In one embodiment, the medical implant device comprises a flexible framework to maintain a luminary space within a vessel, the flexible framework having an inner diameter and outer diameter to radially support a vessel and mechanically or electro-mechanically expand; one or more expansion components positioned within the inner diameter or integral to the flexible framework and capable of structurally configuring said flexible framework into at least a first radial position; and one or more sensors integrated therein and having communication with a remote operable device; wherein the sensors control expansion of the flexible framework into at least a second radial position.

In one embodiment, the medical implant device has one or more expansion components including a matrix of one or more shape memory alloys or shape memory polymers. The expansion components are individually configured into radial halo structures, individually or in combination into an integral network structure. The components of the medical implant device can be biocompatible to avoid any additional complications during the medical procedure.

In another embodiment, one or more expansion components are biodegradable such that any future adjustment of the stent through the electro-mechanical controls does not interfere with the expansion components. Any restenosis corrections can therefore be made through the adjustment of the framework alone without the internal expansion components interfering.

In one aspect, embodiments of the device comprise an activation means for structural reconfiguration of said vessel. The activation means is a communication component to control the mechanical or electro-mechanical operation of the framework. The framework conforms longitudinally or radially with the vessel and allocates a controlled expansion of the vessel walls to permit a sensor controlled flow of fluid through the vessel, while monitoring pressures, temperature, fluid viscosity, and other physiological conditions. Visual optics may also be integrated with the sensors and controls. The remote operable device controls said activation means at any phase of surgery, catherization, and or post-surgical close.

The medical implant device may have one or more expansion components that are temperature activated. The expansion components could, however, be composed of current stent materials, including metallic compositions, nitriles, polymers, and any mixtures thereof.

In one aspect, the one or more expansion components have a fixed expansion coefficient for safety and efficacy of stent positioning.

One embodiment of the medical implant device utilizes a metal flexible framework of mesh. Other embodiments utilize strong polymeric compositions, shape memory alloys and such materials, alone or in combination.

The medical implant device integrates various sensors to provide visualization of anatomical physiology, including monitoring and control of viscosity, fluid flow, temperature or pressure. Various controls, including MEMS devices are embedded with the framework or expansion components to provide ongoing remote control and operation, particularly remote physiological condition of the patient's vascular condition. Nano-scale devices are also capable of being integrated as desired for smaller vascular structures. The sensors may have manual or computerized controls for manual or automated operation from remote central location or from any specially encrypted remote operable device.

The method of using the medical implant device of the invention comprises the steps of: calculating the physiological parameters for positioning said flexible framework in combination with the one or more expansion components inside said vessel; if desirable, compressing the flexible framework for placement inside said vessel; engaging said flexible framework within the vessel through a guided catherized placement or surgical grafting; operating the expansion components to radially expand the vessel to a first location. The flexible framework can then be repeatedly adjusted from a remote location by a remote operable device such that the flexible framework is gradually configured to a second radial position. The controls are reversible so as to prevent prolonged overexpansion of a vessel and vessel damage.

In one aspect, the gradual expansion of the flexible framework can continue into a number of positions. The expansion is also reversibly controlled to provide safe controlled measures and personalized stent expansion. Thus, the step of engaging includes a computerized sensor system to allocate proper pressure and expansion of said vessel. The step of operating the flexible framework is external a surgical interface or post-surgical close.

In another embodiment, the invention is a medical implant system comprising: a flexible tubular framework to maintain a luminary space within a vessel, said flexible tubular framework having an inner diameter and outer diameter to radially support a vessel; at least a first set of expansion components positioned within said inner diameter or integral to said flexible tubular framework and capable of structurally configuring said flexible tubular framework into at least a first radial position; at least second set of expansion components positioned within said inner diameter or integral to said flexible tubular framework and capable of structurally configuring said flexible tubular framework into at least a second radial position; and one or more sensors integrated therein to adjust positioning of said flexible tubular framework into said first radial position and said second radial position. The sensors may be placed in communication with a remote operable device.

In one aspect, the first set of expansion components establish a first radial position at initial placement and a second set of expansion components establish a second radial position at a later timeframe, such that the second radial position is larger than the first radial position. Desirably, the flexible tubular framework comprises a network of cylindrical scaffolds, each having an intraluminal (interior) surface and extraluminal (or exterior) surface. In a two-part coaxial configuration, one cylindrical scaffold expands initially and then a second cylindrical scaffold expands at a second timepoint. Each scaffold comprises its own set of expansion components, yet each is integrally configured into the same uniform framework.

Since visualization may be preferably, one embodiment includes fiber optic components embedded along a surface of the cylindrical scaffold, the fiber optic components including an individual optical sensor for visualization. In another aspect, the medical implant system may include one or more sensors which allow for longitudinal directional expansion or deflection of the flexible tubular framework, including telescopic movement and telescopic visualization of one or more surfaces of said flexible tubular network.

For exemplary purposes only, and not limitation, one embodiment is a flexible polymeric framework with expansion components integrated therein. Any rigid stent structure may be utilized in combination. Any number of sensors, communications devices and controls may be implemented to assist in remote operation of the stent, particularly at a date or time post-surgical close. The implant device transforms the vessel into a desirable shape.

### BRIEF DESCRIPTION OF THE DRAWINGS

The invention is best understood from the following detailed description when read with the accompanying drawing figures. It is emphasized that the various features are not necessarily drawn to scale. In fact, the dimensions may be arbitrarily increased or decreased for clarity of discussion. Further, the below representations may not be drawn to scale.

FIG. 1 is a side view of an illustrative embodiment of the device of the invention.

FIG. 2 is a side view of an illustrative embodiment of the device of the invention.

### DETAILED DESCRIPTION

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention.

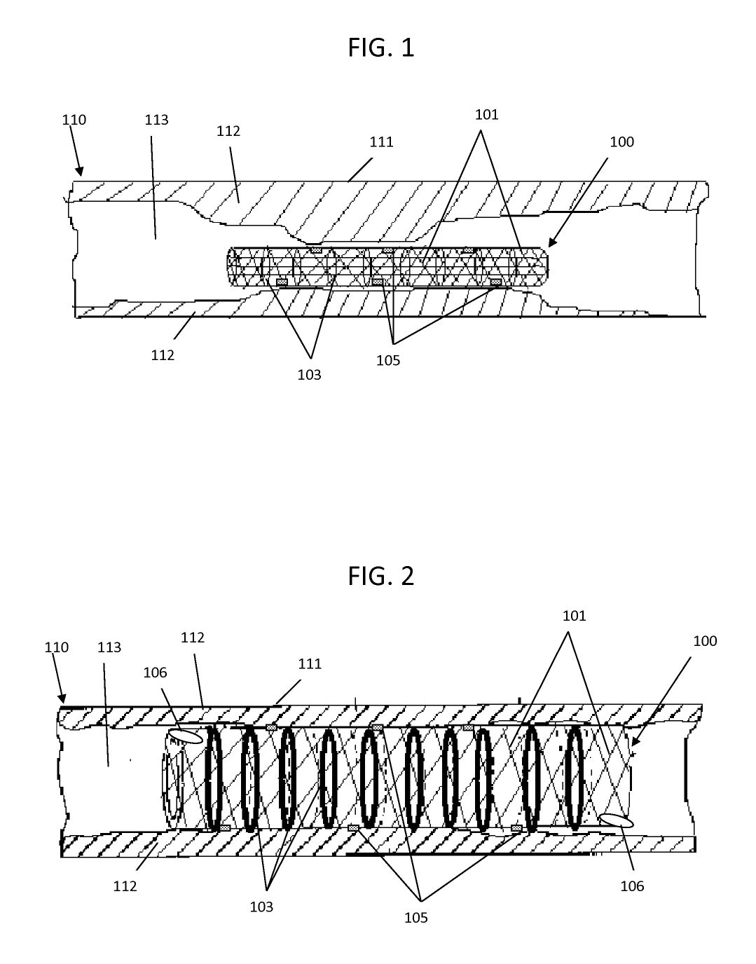

In accordance with one embodiment of the present invention, an illustrative view of a medical implant device 100 within a vessel 110 is shown in FIG. 1. The arterial wall 111 is blocked by cholesterol deposits 112 inside the lumen 113 of the artery 110 to form a blockage which restricts or blocks blood flow, leading to high blood pressure or heart attack. The stent 100 is comprised of an expandable framework structure, or scaffold 101, having a flexible configuration for radial compression to be guided through a lumen 111 of the vessel 110 to a designated treatment site of stenosis. Integral with the scaffold 101 are halo expansion rings 103 comprised of self-expanding shape memory materials (SMM). Various microelectronic sensors 105 (microelectromechanical systems, MEMS 105) are placed upon internal surfaces of the flexible framework 101 which provide remote operation or actuation. The sensors 105 are placed in a manner to avoid any interference with the expansion of the flexible scaffold 101.

Once the stent 100 is in place, the stent is radially expanded into a designated position to increase blood flow through the lumen 113 of the vessel 110 (See FIG. 2). As illustrated in FIG. 2, the halo expansion rings 103 are shape memory polymers which are heated to cause the shape memory polymer to transition to the expanded configuration, thereby deploying the tubular shape of the flexible framework within the target site of the blood vessel or body lumen, or within an aneurysm or stenosed cardiac artery, to a first radial position. A locking mechanism may be included as a safety feature to prevent axial contraction of the scaffold 101. In one aspect, the mechanism is useful when utilizing biodegradable expansion components. Although the scaffold itself will resist recoil or external compression forces, or radial resistive forces, the mechanism ensures than the luminary space of the vessel remains open.

When the MEMS device 105 is activated from a remote device, the scaffold 101 is expanded to a second radial position or various positions. The positioning may be gradual or step increments to be adjusted and controlled by the attending physician. The remote control of the stent scaffold 101 allows a physician to curtail the effects of restenosis and allows a truly minimal invasive procedure. Internal visualization integral with the scaffold 101 through fiber optic components 106 allows the physician to adjust specific individual MEMS devices 105 within the stent, or control all simultaneously. Visualization devices 106 allow the physician to expand the stent by nano to micro measurements to ensure the safety of the patient and efficacy of the device. In one embodiment, the visualization devices are isolated optical components at the perimeter of the scaffold to see any intervening events of restenosis. An alert may be connected to the MEMS device for emergency automation or for contacted an emergency care facility. Any number of sensors with various capabilities may be interconnected with the electrical and mechanical operation of the device of the invention.

In one embodiment, fiber optic members may be utilized in the scaffolding for support, visualization, and/or heating the polymer. In one aspect, the expansion components are polymers having a glass transition temperature (Tg) below body temperature, making the transition from a compressed configuration to an expanded configuration when exposed to body temperature. In another aspect, the polymers may be biodegradable within a short-time period of time, of about six months such that the flexible framework/scaffold 101 remains when the expansion components degrade and thereby eliminating any physiological interference when the scaffold 101 is expanded by a remote control device. In yet another aspect, the expansion components are shape memory alloys, including nitinol or other metallic compositions, such as those regulated for medical applications.

One embodiment utilizes an expansion component made of a first shape memory material (SMM) having a 1^{st} shape transition temperature and a second shape memory material having a 2^{nd} shape transition temperature greater than the first SMM, wherein the first SMM expands to a pre-shape when the first SMM is heated to above the 1^{st} shape transition temperature and the second SMM expands to a pre-shape when heated to above the 2^{nd} shape transition temperature. These materials may be beneficial in determining the expansion within a particular vessel.

For exemplary purposes, and not limitation, the device may take the size and shape as desired to facilitate an increased luminary space within a vessel. The implantable device expands a vascular lumen to increase the blood flow through the vessel. The implantable device is a stent capable of various radial positions and operable by various communications means, including wireless devices.

A number of implementations have been described. Nevertheless, it will be understood that various modifications may be made without departing from the spirit and scope of this document. In particular, SMA materials can be biocompatible and composed of materials currently implemented in the design of stents (for use interior to the vessel). The SMA may have a tubular framework, a plurality of openings between the tubular framework. A woven design of the SMA may provide greater structural support. A metallic composition included with the polymer may be desirable for a more rigid structure. Various angles and shapes of the device may be implemented to position the device. Accordingly, instrumentation for insertion of the device and or use of catheters may be utilized to place the device. Other implementations are within the scope of the following claims.

Further, the use of multiple halo expansion components, alone or in combination, or simultaneously, can facilitate the performance of the device.

Even further, the deployment of micro-devices for in vivo monitoring and on-site treatment are beneficial for minimally invasive procedures in the clinical setting. Thus, any stent could be modified and configured to be remotely operable in vivo using a remote activation system and visualization means.

What is claimed is:

1. A medical implant device comprising:
a flexible tubular framework to maintain a luminary space within a vessel, said flexible tubular framework having an inner diameter and outer diameter to radially support a vessel;
one or more expansion components positioned within said inner diameter or integral to said flexible tubular framework and capable of structurally configuring said flexible tubular framework into at least a first radial position; and
one or more sensors integrated therein and having communication with a remote operable device;
wherein said sensors adjust positioning of said flexible tubular framework into said first radial position.

2. The medical implant device of claim 1, wherein said sensors control expansion of said flexible tubular framework into at least a second radial position.

3. The medical implant device of claim 1, wherein said one or more expansion components is a matrix of one or more shape memory alloys or shape memory polymers.

4. The medical implant device of claim 1, wherein said one or more expansion components is biocompatible or biodegradable.

5. The medical implant device of claim 1, further comprising an activation means for structural reconfiguration of said vessel.

6. The medical implant device of claim 1, wherein said one or more expansion components is temperature activated.

7. The medical implant device of claim 1, wherein said flexible tubular framework is comprised of one or more materials including metals, shape memory materials or alloys, and polymeric compositions, individually or in combination.

8. The medical implant device of claim 1, wherein one or more of said sensors provide visualization of anatomical physiology, including monitoring and control of viscosity, fluid flow, temperature or pressure.

9. The medical implant device of claim 1, wherein one or more of said sensors is a micro-electro-mechanical system (MEMS) or nano-scale device.

10. The method of using a remote control medical implant device, comprising the steps of:
calculating the physiological parameters for positioning a flexible framework in combination with one or more expansion components inside a vessel of a patient;
engaging said flexible framework within said vessel, independently or in combination with said one or more expansion components;
operating said flexible framework from a remote location.

11. The method of claim 10, wherein said step of engaging said one or more expansion components configure said flexible framework into at least a first radial position.

12. The method of claim 10, further comprising a step of providing two or more expansion components having different expansion coefficients.

13. The method of claim 10, wherein said step of operating said flexible framework is configured to reversibly narrow.

14. The method of claim 10, wherein said step of operating is a gradual expansion of said flexible framework into one or more positions.

15. The method of claim 10, wherein said step of engaging includes a computerized sensor system to allocate proper pressure and expansion of said vessel.

16. The method of claim 10, wherein said step of operating said flexible framework is external a surgical interface or post-surgical close.

17. The method of claim 10, wherein said step of engaging includes a balloon expansion mechanism to situate said flexible framework.

18. The medical implant system comprising
a flexible tubular framework to maintain a luminary space within a vessel, said flexible tubular framework having an inner diameter and outer diameter to radially support a vessel;
at least a first set of expansion components positioned within said inner diameter or integral to said flexible tubular framework and capable of structurally configuring said flexible tubular framework into at least a first radial position;
at least second set of expansion components positioned within said inner diameter or integral to said flexible tubular framework and capable of structurally configuring said flexible tubular framework into at least a second radial position; and
one or more sensors integrated therein to adjust positioning of said flexible tubular framework into said first radial position and said second radial position.

19. The medical implant system of claim 18, wherein said sensors are placed in communication with a remote operable device.

20. The medical implant system of claim 18, wherein said second set of expansion components establish said second radial position at a later timeframe, said second radial position being larger than said first radial position.

21. The medical implant system of claim 18, wherein said flexible tubular framework is cylindrical scaffold having a two-part coaxial configuration of a cylinder within a cylinder to include said first set of expansion components and said second set of expansion components.

22. The medical implant system of claim 18, wherein one or more fiber optic components are embedded along a surface of said cylindrical scaffold, said fiber optic components including an individual optical sensor for visualization.

23. The medical implant system of claim 18, wherein said one or more sensors allows for longitudinal directional expansion or deflection of said flexible tubular framework, including telescopic movement and telescopic visualization of one or more surfaces of said flexible tubular network.
